SEQUENCE LISTING (8 sequences)

SEQ ID NO: 1 - DNA, 1583 bp, Homo sapiens
  CDS (199)..(1338)
caggagacaggcttcctccagggtctggagaacccagaggcagctcctcctgagtgctgggaaggactctgggcatcttcagcccttcttactctctgaggctcaagccagaaattcaggctgcttgcagagtgggtgacagagccacggagctggtgtccctgggaccctctgcccgtcttctctccactccccagcatggaggaaggtggtgattttgacaactactatggggcagacaaccagtctgagtgtgagtacacagactggaaatcctcgggggccctcatccctgccatctacatgttggtcttcctcctgggcaccacgggaaacggtctggtgctctggaccgtgtttcggagcagccgggagaagaggcgctcagctgatatcttcattgctagcctggcggtggctgacctgaccttcgtggtgacgctgcccctgtgggctacctacacgtaccgggactatgactggccctttgggaccttcttctgcaagctcagcagctacctcatcttcgtcaacatgtacgccagcgtcttctgcctcaccggcctcagcttcgaccgctacctggccatcgtgaggccagtggccaatgctcggctgaggctgcgggtcagcggggccgtggccacggcagttctttgggtgctggccgccctcctggccatgcctgtcatggtgttacgcaccaccggggacttggagaacaccactaaggtgcagtgctacatggactactccatggtggccactgtgagctcagagtgggcctgggaggtgggccttggggtctcgtccaccaccgtgggctttgtggtgcccttcaccatcatgctgacctgttacttcttcatcgcccaaaccatcgctggccacttccgcaaggaacgcatcgagggcctgcggaagcggcgccggctgctcagcatcatcgtggtgctggtggtgacctttgccctgtgctggatgccctaccacctggtgaagacgctgtacatgctgggcagcctgctgcactggccctgtgactttgacctcttcctcatgaacatcttcccctactgcacctgcatcagctacgtcaacagctgcctcaaccccttcctctatgcctttttcgacccccgcttccgccaggcctgcacctccatgctctgctgtggccagagcaggtgcgcaggcacctcccacagcagcagtggggagaagtcagccagctactcttcggggcacagccaggggcccggccccaacatgggcaagggtggagaacagatgcacgagaaatccatcccctacagccaggagacccttgtggttgactagggctgggagcagagagaagcctggcgccctcggccctccccggcctttgcccttgctttctgaaaatcaggtagtgtggctactccttgtcctatgcacatcctttaactgtcccctgattctgccccgccctgtcctcctctactgcttgattctttctcagaggtttgtggtttaggggaaagagactgggtctacagacctgaccctgcacaagccatttaatctcactcagcctctgt

SEQ ID NO: 2 - protein, 380 aa, Homo sapiens
MEEGGDFDNYYGADNQSECEYTDWKSSGALIPAIYMLVFLLGTTGNGLVLWTVFRSSREKRRSADIFIASLAVADLTFVVTLPLWATYTYRDYDWPFGTFFCKLSSYLIFVNMYASVFCLTGLSFDRYLAIVRPVANARLRLRVSGAVATAVLWVLAALLAMPVMVLRTTGDLENTTKVQCYMDYSMVATVSSEWAWEVGLGVSSTTVGFVVPFTIMLTCYFFIAQTIAGHFRKERIEGLRKRRRLLSIIVVLVVTFALCWMPYHLVKTLYMLGSLLHWPCDFDLFLMNIFPYCTCISYVNSCLNPFLYAFFDPRFRQACTSMLCCGQSRCAGTSHSSSGEKSASYSSGHSQGPGPNMGKGGEQMHEKSIPYSQETLVVD

SEQ ID NO: 3 - DNA, 21 bp, Artificial
  5-APJ-Primer
tcccaggagtaaaagccaagc

SEQ ID NO: 4 - DNA, 20 bp, Artificial
  3-APJ-Primer
ccttcaagggtcctgtcagc

SEQ ID NO: 5 - DNA, 34 bp, Artificial
  APJ-Primer
agaggttgtttttgccaagaatcacagaatgtta

SEQ ID NO: 6 - DNA, 20 bp, Artificial
  5-beta-Actin-Primer
tccaccttccagcagatgtg

SEQ ID NO: 7 - DNA, 21 bp, Artificial
  3-beta-Actin-Primer
ctagaagcatttgcggtggac

SEQ ID NO: 8 - DNA, 29 bp, Artificial
  beta-Actin-Primer
atcagcaagcaggcagtatgacgagtccg

(19) United States
(12) Patent Application Publication
(10) Pub. No.: US 2005/0075275 A1
(43) Pub. Date: Apr. 7, 2005

(54) Regulation of the apj receptor for use in the treatment or prophylaxis of cardiac diseases

(75) Inventors: Barbara Albrecht, Wulfrath (DE); Horst-Peter Antonicek, Bergisch Gladbach (DE); Peter Ellinghaus, Wuppertal (DE); Erwin Bischoff, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Aktiengesellschaft, Leverkusen (DE)

(21) Appl. No.: 10/486,061

(22) PCT Filed: Jul. 24, 2002
(86) PCT No.: PCT/EP02/08242

(30) Foreign Application Priority Data
Aug. 6, 2001 (DE) 101 38 569.2

(51) Int. Cl. A61K 38/00; A61K 31/21

(52) U.S. Cl. 514/2; 514/509

(57) ABSTRACT

The invention relates to the use of APJ receptor agonists for producing a medicament for the treatment and/or prophylaxis of coronary heart diseases, in particular stable and unstable angina pectoris, acute myocardial infarction, myocardial infarction prophylaxis, sudden heart death, heart failure, and high blood pressure and the sequelae of atherosclerosis.

The invention relates to the use of APJ receptor agonists for producing a medicament for the treatment and/or prophylaxis of coronary heart diseases, in particular stable and unstable angina pectoris, acute myocardial infarction, myocardial infarction prophylaxis, sudden heart death, heart failure, and high blood pressure and the sequelae of atherosclerosis.

As a ceaselessly working hollow muscle, the heart requires a particularly intensive supply of oxygen to cover its energy requirements. Interferences with supply therefore relate primarily to oxygen transport, which may be inadequate if the adaptability of the blood flow is reduced. An increase in oxygen consumption can be covered only by an increase in the blood flow to the heart.

In coronary heart diseases such as stable and unstable angina pectoris, heart failure, myocardial infarction, sudden heart death, and the sequelae of atherosclerosis, an adequate blood flow to parts of the cardiac tissue is no longer ensured, and tissue ischemias occur, leading to necrosis and apoptosis in the affected areas. This results in myocardial dysfunction which may develop as far as heart failure.

Therapeutic methods and active ingredients which improve coronary blood flow and thus the oxygen supply, but also those which reduce the oxygen consumption, are suitable for treating symptoms of the abovementioned disorders.

These include dilatation of larger coronary vessels, reduction in the extravascular component of the coronary resistance, reduction of the intramyocardial wall tension, and dilatation of the arteriolar resistance vessels in the systemic circulation.

Substances and methods leading to an increase in the coronary flow in the heart and/or to a reduction in blood pressure can be utilized therapeutically (Forth, Henschler, Rummel; Allgemeine und spezielle Pharmakologie und Toxikologie; Urban & Fischer Verlag (2001), Munich).

The effects described above-can be controlled by stimulation of G protein-coupled receptors. G protein-coupled receptors are 7 transmembrane domain proteins whose activation lead to the activation of various G proteins. The activated G proteins in turn are able to activate or inactivate various other signaling systems and thus lead to induction of the mechanisms described above. The G proteins are differentiated into Gαs (adenylyl cyclase activating), Gαi (adenylyl cyclase inhibiting) and Gαq (increase in IP3).

The DNA sequence which codes for the human APJ receptor is shown in SEQ ID NO: 1 in the sequence listing. The amino acid sequence of the human APJ receptor is shown in SEQ ID NO: 2 in the sequence listing. The APJ receptor is a Gαi coupled receptor (O'Dowd et al., Gene, 136 (1993) 355-360) which is activated inter alia by its natural ligand apelin (Tatemoto et al., Biochem. Biophys. Res. Commun., 251 (1998) 471-476). The receptor shows 30% homology with the AT1 (angiotensin) receptor, but is not activated by angiotensin.

The APJ receptor of the rat (described in WO0068250) and the mouse (described in WO0068244) is expressed ubiquitously in many regions of the brain and in other tissues (Hosoya et al., The Journal of Biological Chemistry, 275 (2000) 21061-21067; O'Carroll et al., Biochimica et Biophysica Acta, 1492 (2000) 72-80). In humans, expression of the receptor is described in the spleen, thymus, prostate, testis and the digestive tract (Edinger et al., Journal of Virology, 72 (1998) 7934-7940).

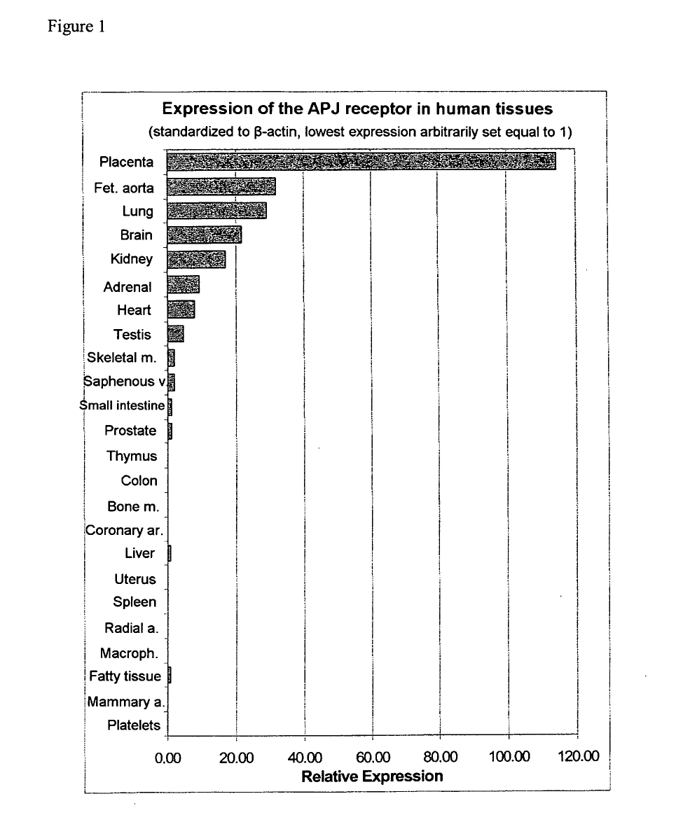

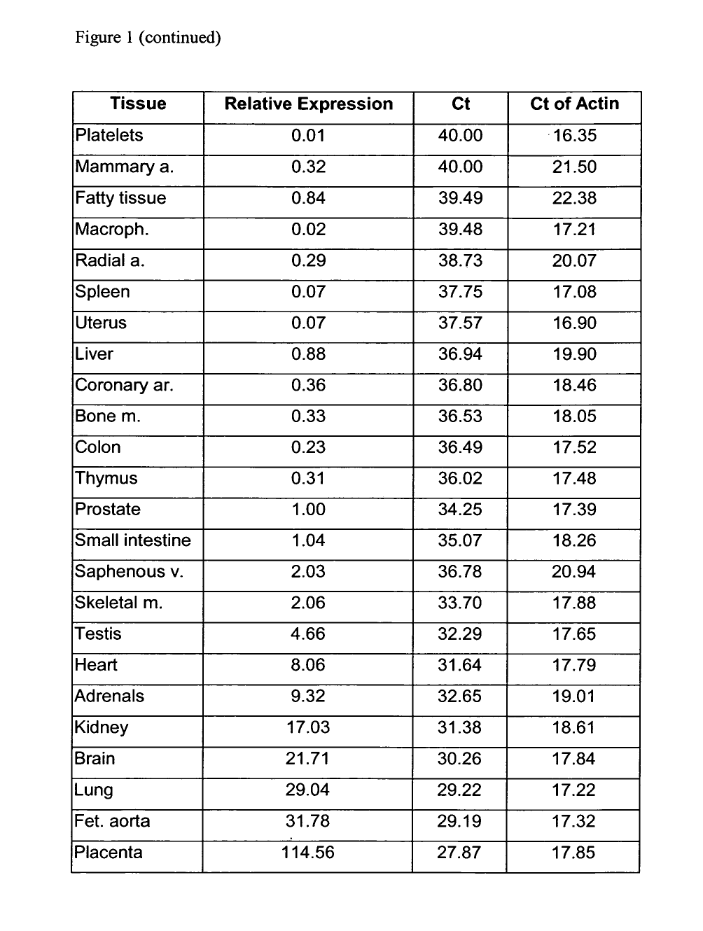

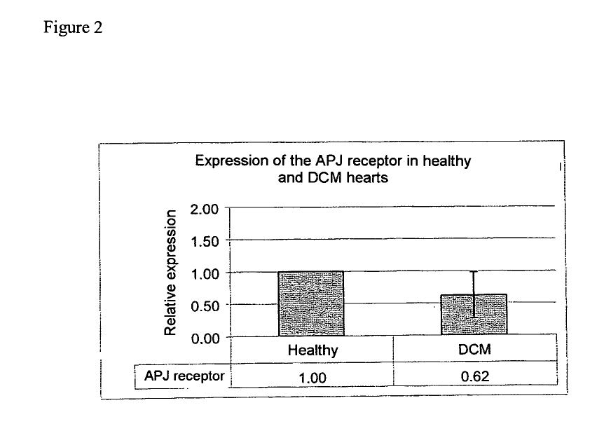

It has surprisingly now been found in quantitative analysis of human APJ receptor mRNA expression that expression of the receptor is restricted to a few tissues and there is pronounced expression of the receptor in the human heart (FIG. 1). It has additionally been found that the APJ receptor occurs both in healthy and in DCM hearts. (DCM=dilated cardiomyopathy) (FIG. 2). Since expression of the human APJ receptor in the diseased heart is a condition for the use of active ingredients for stimulating the receptor in patients with coronary heart diseases, this result creates the basis for a new approach to therapy. A decrease in receptor expression, as is known, for example, for the β1 adrenoreceptor in heart failure (Chakraborti et al., Cellular Signaling, 12 (2000) 499-513), would mean that therapeutic stimulation is ruled out.

It is known that systemic administration of the natural ligand apelin in rats leads to a reduction in blood pressure without a change in the heart rate (Lee eta al., Journal of Neurochemistry, 74 (2000) 34-41; Tatemoto et al., Regulatory Peptides 99 (2001) 87-92). Direct administration of the ligand in the rat brain brings about the inhibition of water uptake in dehydrogenated rats without having an effect on blood pressure (Reaux et al., Journal of Neurochemistry, 77 (2001) 1085-1096). Accordingly, the reduction in blood pressure appears to be induced by direct stimulation of the receptor in vessels.

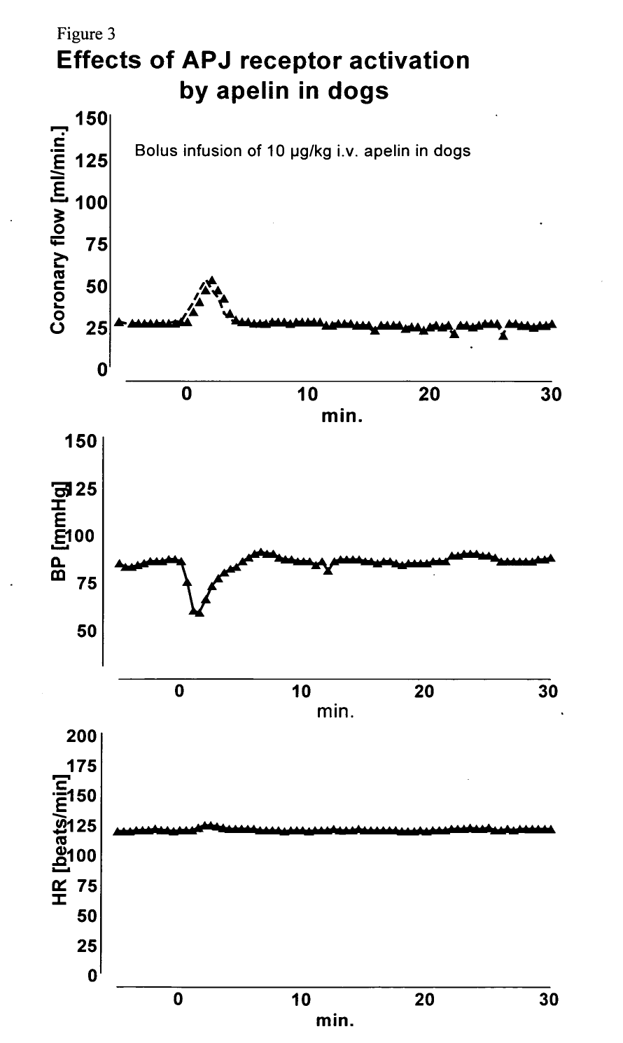

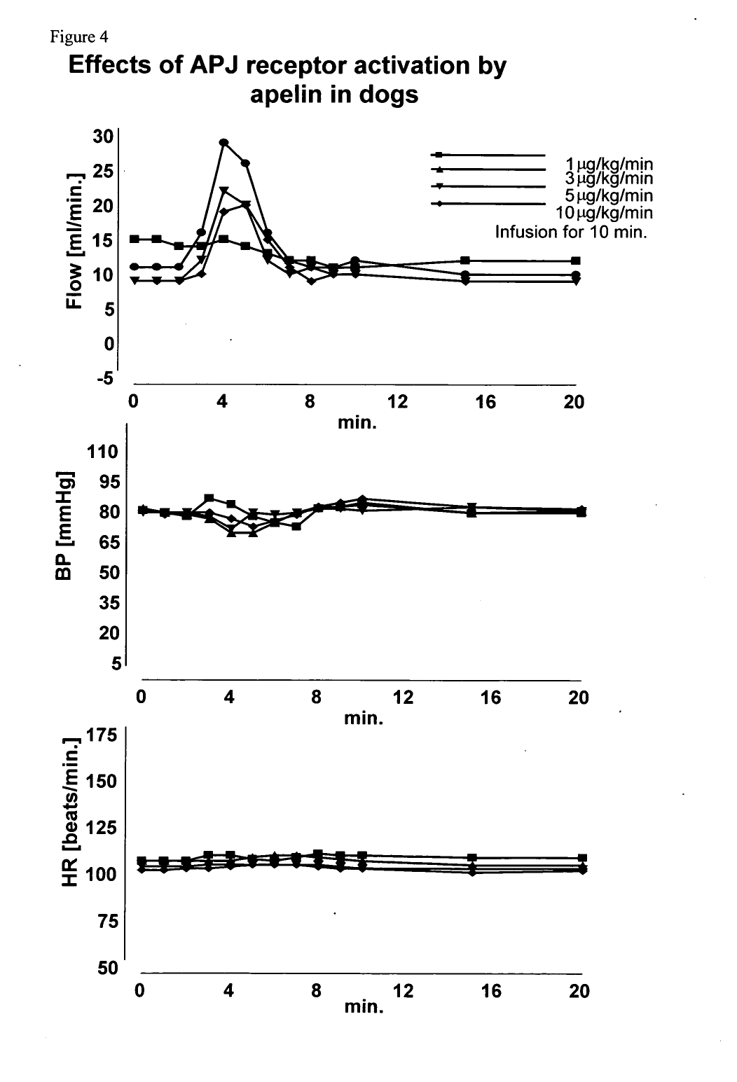

Surprisingly, systemic administration of apelin, both as bolus and as continuous infusion, in dogs brings about a concentration-dependent, very marked increase in coronary flow without changing the heart rate (FIGS. 3, 4). This increase in coronary flow goes far beyond the reflex extent of the reduction in blood pressure which is likewise observed.

On the basis of this new result, we concluded that substances which stimulate the APJ receptor can, because of the increase, resulting therefrom, in the coronary flow, be employed for the treatment and/or prophylaxis of stable and unstable angina pectoris, acute myocardial infarction, myocardial infarction prophylaxis, heart failure, sudden heart death, and high blood pressure and the sequelae of atherosclerosis in humans.

The present invention therefore relates to the use of APJ receptor agonists for producing a medicament for the treatment and/or the prophylaxis of the abovementioned diseases.

Agonists for the purposes of the invention are all substances which bring about a stimulation of the biological activity of the receptor. Particularly preferred agonists are nucleic acids including locked nucleic acids, peptide nucleic acids and “spiegelmers”, proteins including antibodies and low molecular weight substances, and very particularly preferred agonists are low molecular weight substances.

The stimulation can be measured for example in the APJ stimulation test described below. In this connection, there is stimulation of the APJ receptor if a decrease of at least 10% in a cAMP level which is elevated through stimulation of adenylate cyclase is measured in the test, it being possible for the stimulation to take place inter alia through forskolin or via adrenergic receptors.

APJ receptor agonists can be tested on recombinant cell lines which contain the human APJ receptor.

APJ agonists preferred in this connection are those which activate with an EC₅₀ of 1 μM, preferably less than 0.1 μM, in the APJ stimulation test indicated below.

The APJ receptor agonists of the invention are preferably unable to cross the blood/brain barrier and have systemic but no central effects.

### DESCRIPTION OF THE FIGURES

1.) Relative expression of the human APJ receptor in human tissue

2.) Comparison of the relative expression of the human APJ receptor in healthy and DCM (dilated cardiomyopathy) cardiac tissue

3.) Effect of in vivo administration of apelin as bolus on blood pressure (BP), heart rate (HR) and coronary flow in dogs

4.) Effect of infusion of various apelin concentrations for 10 minutes on blood pressure (BP), heart rate (HR) and coronary flow in dogs

### APJ STIMULATION TEST

The effect of apelin and other possible APJ agonists is tested on a CHO cell line which stably expresses the complete open reading frame of the human receptor gene as recombinant protein. On activation of this cell line with forskolin there is an increase in the internal cAMP level. This increase can be prevented by simultaneous or previous administration of apelin via stimulation of the recombinantly expressed APJ receptor and the inhibition, resulting therefrom, of adenylyl cyclase.

1500 CHO cells/well are seeded in 384-well plates (Greiner) in DMEM medium (Gibco) with 10% FCS (fetal calf serum, Gibco) and incubated at 37° C. and 5% CO₂ for 2 days. The cells are incubated with apelin dilutions (serial dilutions typically of 10⁻¹³-10⁻⁷ M) and then stimulated with 10⁻⁵ M forskolin. The cAMP level is determined with the aid of the cAMP screen kit [Tropix (PE Biosystems)] in accordance with instructions, and the affect of apelin is represented as percentage inhibition of the maximum cAMP elevation after stimulation with forskolin. The EC₅₀ of the effect of apelin is the value at which 50% of the forskolin signal are inhibited.

An EC₅₀ of 10⁻¹¹ M is determined for apelin.

### Investigations of APJ Receptor Expression in Humans

The relative expression of the APJ receptor in human tissues is measured by quantifying the mRNA by means of the real-time polymerase chain reaction (so-called TaqMan-PCR, Heid et al., Genome Res 6 (10), 986-994). Compared with conventional PCR, the real-time PCR has the advantage of more accurate quantification through the introduction of an additional, fluorescence-labeled oligonucleotide. This so-called probe comprises the fluorescent dye FAM (6-carboy-fluorescein) at the 5′ end and the fluorescence quencher TAMRA (6-carboxy-tetra-methylrhodamine) at the 3′ end. During the polymerase chain reaction in the TaqMan PCR, the fluorescent dye FAM is cleaved off the probe by the 5′-exonuclease activity of Taq polymerase, and thus the previously quenched fluorescence signal is retained. The cycle number at which the fluorescence intensity is about 10 standard deviations above the background fluorescence is recorded as the so-called threshold [treshold cyle (Ct)].

The starting material used for the PCR is cellular RNA obtained commercially (from Clontech). In the case of human hearts, small pieces (about 0.5 g) of explanted hearts are isolated from patients with dilated cardiomyopathy (obtained from Deutsches Herzzentrum Berlin), and the total RNA is isolated therefrom using RNaesy columns (from Qiagen) in accordance with instructions. In each case 1 μg of total RNA from each tissue is reacted with 1 unit of DNase I (from Gibco) at room temperature for 15 min to remove genomic DNA contamination. The Dnase I is inactivated by adding 1 μl of EDTA (25 mM) and subsequently heating at 65° C. (10 min).

Subsequently, cDNA synthesis is carried out in the same reaction mixture in accordance with the instructions for the “SUPERSCRIPT-II RT cDNA synthesis kit” (from Gibco), and the reaction volume is made up to 200 μl with distilled water. For the PCR, 7.5 μl of mixture of primer and probe, and 12.5 μl of TaqMan reaction solution [Universal Master Mix (from Applied Biosytems] are added to 5 μl portions of the diluted cDNA solution. The final concentration of the primer is 300 nM in each case, and that of the probe is 150 nM. The sequence of the forward and reverse primers for the APJ receptor is: 5′-TCCCAGGAGTAAAAGCCAAGC-3′ and 5′-CCTTCAAGGGTCCTGTCAGC-3′, and the sequence of the fluorescent probe is 5′-6FAM-AGAGGTTGTTTTTGCCAAGAATCACAGAATGTTA-TAMRA-3′. The PCR takes place in an ABI prism SDS 7700 apparatus (from Applied Biosystems) in accordance with the manufacturer's instructions. 40 cycles are carried out. The Ct (see above) obtained for the APJ receptor for each tissue corresponds to the cycle in which the fluorescence intensity of the liberated probe reaches 10 times the background signal. Thus, a lower Ct means an earlier start of replication, i.e. more mRNA present in the original sample. To compensate for possible variations in the cDNA synthesis, the expression of a so-called “housekeeping gene” is also analyzed in all tissues investigated. Expression of this gene should be approximately the same in all the tissues. For standardizing APJ receptor expression, β-actin is used for this purpose. The sequence of the forward and reverse primers for β-actin is 5′-TCCACCTTCCAGCAGATGTG-3′, and 5′-CTAGAAGCATTTGCGGTGGAC-3′, and the sequence of the probe is 5′-6FAM-ATCAGCAAGCAGGCAGTATGACGAGTCCG-TAMRA-3′. The data are analyzed by the so-called dCt method in accordance with the instructions for the ABI prism SDS 7700 (from Applied Biosystems). For graphical representation of the tissue distribution of the APJ receptor, the tissue with the lowest relative expression is arbitrarily set equal to 1 (FIG. 1) and all other tissues are standardized thereto. Not depicted are tissues having a Ct of >35. For analyzing the expression of the APJ receptor in the hearts, the average relative expression from the healthy control hearts is set equal to 1 and compared with the average from the DCM hearts (FIG. 2).

The human APJ receptor expression data show that the receptor has a Ct of 31.64 in the heart, corresponding to an average expression of the receptor in the heart. Comparison of the amount of mRNA in healthy and DCM tissue shows that there is no significant reduction in receptor expression in DCM hearts, and thus stimulation of the receptor by agonists can also be utilized therapeutically for patients with coronary heart diseases.

### In Vivo Investigations of the Stimulation of the APJ Receptor by Apelin

Adult FBI (Foxhound-Beagle-Irish-Setter) dogs (20-30 kg) are initially anesthetized with thiopental Na (Trapanal, Byk-Gulden) 20-30 mg/kg i.v. Alcuronium chloride (Alloferin, Roche) 0.1 mg/kg is initially given i.v. for muscle relaxation. The animals are intubated and ventilated with a ⅓ O₂/N₂O mixture using an Engström respiratory pump with 15-18 breaths per min and a volume of 18-24 ml/kg. The ventilation is set accurately according to the arterial partial pressure of CO₂, maintaining an average pCO₂ of 35-45 mmHg. The maintenance anesthesia is done with isoflurane (Baxter) 1.5-3%. The body temperature is kept at 38° C.±0.1° C. The arterial blood pressure is measured via a catheder in the femoral artery. A thoracotomy is performed at the fifth intercostal space on the left side. The lung is retracted and fixed, and the pericardium is incised. A proximal section of the LAD (left coronary artery) distal to the first diagonal branch is exposed, and a calibrated electromagnetic flow probe (Gould Statham, model SP7515) is placed around the vessel and connected to a flowmeter (Statham, model SP-2202). A mechanical occluder is attached distal of the flow probe in such a way that no branches lie between flow probe and occluder.

Blood is taken and substances are administered through a catheder in the femoral vein. A peripheral ECG is recorded with subcutaneously fixed needles. A microtip pressure manometer (Millar model PC-350) is pushed through the left atrium in order to measure the left ventricular pressure. Measurement of the heart rate is controlled by the R wave of the ECG. The hemodynamic parameters and the coronary flow are recorded on a multichannel recorder throughout the experiment.

The experiment is started after a stabilization time of 1 hour. Either 10 μg/kg apelin in physiological saline are injected as i.v. bolus, or various apelin concentrations (1, 3, 5, 10 μg/kg/min) are infused for 10 min. The heart rate, blood pressure and coronary flow are recorded and analyzed.

Both administration of bolus and infusion of apelin brings about a significant increase in the coronary flow in dogs despite a reduction in the blood pressure (FIG. 3). Apelin infusion shows a dose-dependent increase in coronary flow, with the maximum being reached at 3 μg/kg/min (FIG. 4).

### APJ Receptor Agonist Formulations

The APJ receptor agonists can be converted in a known manner into conventional formulations such as tablets, coated tablets, pills, granules, aerosols, syrups, emulsions, suspensions and solutions using inert, nontoxic, pharmaceutically suitable carriers or solvents. In these cases, the therapeutically active compound should in each case be present in a concentration of about 0.5 to 90% by weight of the complete mixture, i.e. in amounts which are sufficient to reach the stated dose range.

The formulations are produced for example by extending the active ingredients with solvents and/or carriers, where appropriate using emulsifiers and/or dispersants, it being possible for example when water is used as diluent where appropriate to use organic solvents as auxiliary solvents.

Administration takes place in a conventional way, preferably orally, transdermally, intravenously or parenterally, in particular orally or intravenously. However, it can also take place by inhalation through the mouth or nose, for example with the aid of a spray, or topically via the skin.

It has generally proved advantageous to administer amounts of about 0.001 to 10 mg/kg, on oral administration preferably about 0.005 to 3 mg/kg, of body weight to achieve effective results.

It may nevertheless be necessary where appropriate to deviate from the stated amounts, in particular as a function of the body weight or of the mode of administration, of the individual behavior toward the medicament, the nature of its formulation and the time or interval over which administration takes place. Thus, it may be sufficient in some cases to make do with less than the aforementioned minimum amount, whereas in other cases the stated upper limit must be exceeded. When larger amounts are administered, it may be advisable to divide them into a plurality of single doses over the day.

1. A method for treating coronary heart diseases, high blood pressure and the sequelae of atherosclerosis, comprising administering to a patient in need thereof an effective amount of an APJ receptor agonist.

2. The method of claim 1, where the coronary heart diseases are stable and unstable angina pectoris, acute myocardial infarction, myocardial infarction prophylaxis, sudden heart death and heart failure.

3. The method as claimed in claim 1, where the APJ receptor agonist has an EC₅₀ of less than 1 μM.

4. The method as claimed in claim 1, where the human APJ receptor agonist has an EC₅₀ of less than 100 μM.
